(19) United States
(12) Patent Application Publication
(10) Pub. No.: US 2008/0014282 A1
(43) Pub. Date: Jan. 17, 2008

(54) Therapeutic, Nutraceutical and Cosmetic Applications for Eggshell Membrane and Processed Eggshell Membrane Preparations

(75) Inventors: Frank Daniel Long, Neosho, MO (US); Randall Gene Adams, Carthage, MO (US); Dale Paul DeVore, Chelmsford, MO (US); Marshall Robert Franklin, Carthage, MO (US)

Correspondence Address: HOFFMANN & BARON, LLP, 6900 JERICHO TURNPIKE, SYOSSET, NY, 11791, US

(73) Assignee: NEW LIFE RESOURCES, LLC, Carthage, MO (US)

(21) Appl. No.: 11/861,438

(22) Filed: Sep. 26, 2007

Related U.S. Application Data
(62) Division of application No. 10/797,747, filed on Mar. 10, 2004.
(60) Provisional application No. 60/453,891, filed on Mar. 12, 2003.
(60) Provisional application No. 60/488,538, filed on Jul. 18, 2003.
(60) Provisional application No. 60/501,364, filed on Sep. 9, 2003.

(51) Int. Cl. A61K 31/728; A61K 31/726; A61K 31/737; A61K 35/12; A61K 35/32; A61K 35/56; A61K 35/60; A61P 3/02

(52) U.S. Cl. 424/547; 424/548; 424/581; 514/54; 514/62

(57) ABSTRACT

The present invention relates to therapeutic, cosmetic, and nutraceutical applications for eggshell membrane, processed eggshell membrane preparations and eggshell membrane isolates. The invention is directed to treating a mammal that will benefit from the administration of naturally occurring material found in eggshell membrane, processed eggshell membrane, eggshell membrane isolates and combinations thereof by administering to the mammal a composition containing eggshell membrane, processed eggshell membrane, eggshell membrane isolates and combinations thereof, alone or in combination with other active ingredients.

### PRIORITY APPLICATIONS

This application claims the benefit of U.S. Provisional Application Nos. 60/453,891 filed on Mar. 12, 2003; 60/488,538 filed on Jul. 18, 2003; and 60/501,364 filed on Sep. 9, 2003.

### FIELD OF THE INVENTION

The present invention relates to therapeutic, cosmetic, and nutraceutical applications for eggshell membrane, processed eggshell membrane preparations and eggshell membrane isolates.

### BACKGROUND

Eggshell membrane is composed of two individual membranes between egg albumin and eggshell. The membranes are primarily comprised of protein fibers. The fibers appear to be a network or scaffold predominantly containing Type I collagen fibers that are encapsulated in a continuous mantle of proteoglycans and other macromolecules.¹ The thickness of the two membranes ranges from 73-114 μm in eggs from White Leghorn and New England pullets.² The outer membrane has a thickness ranging from 53.2 μm to 65.5 μm in White Leghorn eggs while the inner membrane ranges form 19.5 μm to 24.3 μm.

Britton and Hale³ reported that the proteins of shell membranes exhibited a high content of arginine, glutamic acid, methionine, histidine, cystine, and proline. Baker and Balch⁴ and Harris, et. al.⁵ found that eggshell membranes also contained hydroxyproline, hydroxylysine, and desmosine. Subsequent studies by Wong et. al.⁶ demonstrated the presence of Type I and Type V collagen in eggshell membrane. Arias, et. al.⁷ subsequently identified the presence of Type X in eggshell membrane and postulated that Type X collagen functions to inhibit mineralization and establishes a zone protected from mineral deposition.⁸

Eggshell membrane also has been shown to contain acid glycosaminoglycans including dermatan sulfate and chondroitin-4-sulfate⁹. Picard, et. al.,¹⁰ isolated and characterized sulfated glycoproteins from eggshell membrane. Glycoproteins included hexosamines, hexoses, and fucose. More recently, significant amounts of hyaluronic acid have been detected in eggshell membrane (Patent Pending, DeVore, D. P., et. al.). Other components identified in eggshell membrane include ovotransferrin¹¹, desmosine and isodesmosine¹², lysyl oxidase¹³, and lysozyme¹⁴.

The authors of the present patent application have conducted a composition analysis of calcium-free eggshell membrane (devoid of eggshell) finding a high content of protein and significant quantities of glucosamine, chondroitin, and hyaluronic acid. The amino acid profile of eggshell membrane demonstrated high levels of aspartic acid, glutamic acid, proline, arginine, and cystine.

Eggshell membranes are examples of collagenous structures that support development. The most evident function of the membrane is to act as a supporting structure for the egg prior to deposition of a calcified shell and to provide an organic matrix for the deposition of calcium in eggshell formation. The membrane might also be inductive to the developing embryo via secretion of growth factors. The proposed inductive characteristics of eggshell membrane along with its high protein content and significant quantities of glucosamine, chondroitin sulfate, and hyaluronic acid support its potential utility in therapeutics, cosmetics, and nutraceuticals.

Compositions, isolates, and proteins from other natural sources have been previously described for therapeutic, cosmetic, and nutraceutical applications. For example, processed antler powder has been suggested for use in nutraceutical compositions containing such powder. The powder is placed in nutraceutical capsules, formed into tablets or used as an ingredient in nutritional bars or beverages. Methods of preparing lipid fractions from sea cucumbers that can be used as nutraceutical supplements to ameliorate immune responses have also been suggested. The isolates are identified as being useful in the treatment of allergic diseases, inflammatory diseases, and hyperproliferative skin diseases. The preparations may be administered orally or formulated into a topical lotion. Others have disclosed the preparation of milk protein hydrolysates and applications for use in cosmetic formulations. The hydrolysates are described as having skin hydrating properties and wound healing characteristics.

Additional examples of materials derived from natural sources, that have been suggested as therapeutic ingredients, include a protein hydrolysate prepared from poultry feet and processed into a powder or gel for use treating burns or for regrowing muscle, skin, and nerve tissues; a method for treating diseased or injured abraded, lacerated, or ulcerated tissue by applying a composition containing sucrose, gelatin, and water; a method and agent for treating inflammatory disorders of the gastrointestinal tract by administering D-glucosamine hydrochloride in solid or liquid form; and compositions containing amino sugars, such as glucosamine, and a glycosaminoglycan, such as chondroitin, to protect, treat and repair connective tissue.

Other natural materials that have been suggested for use in cosmetics include a cosmetic formulation containing unsaponified lipids extracted from avocado seeds to treat stretch marks, keratosis, and other skin lesions; a cosmetic composition which prevents aging of skin that contains an extract of the aerial part of Cichorium intybus L; a therapeutic and cosmetic composition for use as a hand cream, anti-wrinkle cream, sunscreen cream, moisturizing lotion and deodorant containing an active ingredient obtained from snails; cosmetic compositions containing extracts from the Caribbean Sea Whip (pseudoterogorgia elisabethae); cosmetic skin care compositions containing chick pea extract; cosmetic formulations containing marama bean extract; and cosmetic compositions containing Type V telopeptide collagen prepared from marine invertebrates.

Other oral and topical compositions derived from natural sources for protection, treatment, and repair of connective and skin tissues have been disclosed. For example, an oral and injectable composition comprised of glucosamine, chondroitin sulfate, hydrolyzed or native collagen, sodium hyaluronate, chelated manganese ascorbate, and L-malic acid. The composition acts as a chondroprotective agent, enhances chondrocyte synthesis, healing wounds, and maintaining healthy tissue. Compositions containing glucosamine, chondroitin sulfate, and optionally manganese ascorbate have also been suggested. The topical application or injection of 40-55 wt. % chondroitin sulfate to protect joint cells, reduce aseptic inflammation, and preserve human and animal cells in vitro has also been described. Topical preparations for improving wound healing have also been suggested which include a suspension of collagen and a glycosaminoglycan.

Although certain components (that have now been identified to be present in significant quantities in eggshell membrane, i.e., collagen, glucosamine, chondroitin sulfate, and hyaluronic acid), have been disclosed as being useful in therapeutic, cosmetic, and nutraceutical applications, the use of processed eggshell membrane and/or eggshell membrane isolates in accordance with the present invention have not been previously described.

U.S. Pat. No. 3,194,732 to Neuhauser discloses the use of eggshell membrane and processed eggshell membrane as mechanical barriers or membranes for assisting the healing of skin-denuded areas of the body. It is disclosed that the eggshell membrane can be mechanically processed into dry, finely divided particles or powder. The powder is then applied to a fiber felt carrying sheet to form a layered structure having an outer layer of the eggshell membrane powder. This patent, however, does not disclose a processed eggshell membrane composition for use as an orally or topically administered therapeutic, cosmetic or nutraceutical. Moreover, it does not disclose an isolate of eggshell membrane, a composition containing processed eggshell membrane and/or an eggshell membrane isolate in combination with other active ingredients, or the use of an isolate of eggshell membrane in combination with processed eggshell membrane.

Thus, it has not been previously reported to use processed eggshell membrane and/or eggshell membrane isolates as nutraceuticals to protect, treat, and repair of connective tissues, or to reduce joint pain related to osteoarthritis, rheumatoid arthritis or other joint disorders. Further, it has not been previously reported to use processed eggshell membrane and/or eggshell membrane isolates as natural components of cosmetic formulations to improve skin hydration and treat skin conditions, or to use eggshell membrane isolates and combinations of processed eggshell membrane and eggshell membrane isolates for improving wound healing of skin lacerations, lesions, and ulcerations.

Moreover, although some of the components, which have now been identified to be present in significant quantities in eggshell membrane, i.e., collagen, glucosamine, chondroitin sulfate, and hyaluronic acid, have been obtained from other natural sources, as discussed above, these known sources typically contain lower levels of the target component(s) or require significant processing to isolate the desired component(s) in the required purity. This results in a relatively high cost for the additional processing needed to obtain a useable product.

Therefore, there is a need for a new source of active components useful for the therapeutic, cosmetic and nutraceutical applications discussed above, which naturally contains higher concentrations of these active ingredients and/or requires less processing to provide a useful composition.

### SUMMARY OF THE INVENTION

The present invention describes the preparation and use of eggshell membrane, processed eggshell membrane and eggshell membrane isolates for therapeutic, cosmetic and nutraceutical applications. These applications take advantage of the proposed inductive characteristics of eggshell membrane along with its high protein content and significant quantities of collagen, glucosamine, chondroitin sulfate, and hyaluronic acid; and/or the unique combination of components, of the eggshell membranes, which has uses in therapeutic, cosmetic and nutraceutical applications.

The inventors have investigated eggshell membrane, which was cleanly separated from egg and eggshell, by analyzing for amino acid profiles, glucosamine, chondroitin, and hyaluronic acid. Hyaluronic acid content was measured using three different assays as follows: (1) colorimetric measurement for uronic acid using a modified carbazole reaction¹⁵—uronic acid composes about 50% of the hyaluronic acid molecule. (2) Assay using an ELISA test prepared by Corgenix, Inc.—this enzyme-linked binding protein assay uses a capture molecule known as hyaluronic acid binding protein (HABP) to measure hyaluronic acid concentration. (3) Measurement by size exclusion chromatography using the method of Armstrong and Johns.¹⁶

The first assay method (discussed above) was used to measure hyaluronic acid in aqueous extracts of crude eggshell membranes and in enzyme hydrolysates of eggshell membranes. Results indicated hyaluronic acid concentrations ranging from 0.1% to more than 2% in tested samples. It is suspected that these uronic acid concentrations are lower than expected due to interference of the colorimetric reactions by protein contaminants. More sensitive and specific ELISA assays indicated significantly higher levels of hyaluronic acid in eggshell membrane samples. Concentrations ranged from 5% to more than 10% on a wet weight basis. Measurement using size exclusion chromatography confirmed the presence of hyaluronic acid.

It is believed that these findings are the first reporting of significant quantities of hyaluronic acid, glucosamine and chondroitin in eggshell membrane. Concentrations of glucosamine and chondroitin were 10% and 9% of wet membrane weight, respectively.

The hyaluronic acid, glucosamine, chondroitin, and collagen components can be contained, without further isolation and purification, in “raw” eggshell membrane or mechanically processed eggshell membrane. The hyaluronic acid isolate can also be derived from the eggshell membrane by extracting a hyaluronic acid fraction from the eggshell membrane. In one embodiment, the hyaluronic acid fraction is purified to provide a cosmetic grade or pharmaceutical grade hyaluronic acid. Likewise, the collagen isolates can be derived from eggshell membrane by extracting collagen fractions from the eggshell membrane.

In one embodiment, the invention is directed to a composition for use in cosmetics containing a naturally occurring cosmetically active material derived from eggshell membrane. The cosmetically active material can be selected from the group consisting of mechanically processed eggshell membrane, eggshell membrane hydrolysates, eggshell membrane isolates and combinations thereof. The eggshell membrane hydrolysate or isolate can be in liquid, semi-solid or solid form.

In one embodiment, the eggshell membrane hydrolysate or isolate is rich in a naturally occurring component selected from the group consisting of a hexosamine, chondroitin sulfate, hyaluronic acid, collagen, other proteins and combinations thereof. The eggshell membrane hydrolysate or isolate can be rich in a specific type of collagen, other proteins or combination of specific types of collagen and protein. The eggshell membrane hydrolysate or isolate can also include specific ratios of the naturally occurring components or combinations of the components.

In one embodiment, the eggshell membrane hydrolysate or isolate contains at least about 0.2% hyaluronic acid. Preferably, hyaluronic acid is present in an amount of about 0.2 to about 10 wt %, more preferably, about 0.5 to about 5 wt %, and, most preferably, about 1 to about 5 wt %, based on the weight of the hydrolysate or isolate.

In another embodiment, the eggshell membrane hydrolysate or isolate contains at least about 1% collagen. Preferably, collagen is present in an amount of about 1 to about 40 wt %, more preferably, about 3 to about 25 wt %, and, most preferably, about 5 to about 20 wt %, based on the weight of the hydrolysate or isolate.

It is also contemplated that the composition can include combinations of different isolates, combinations of different eggshell membrane powders, or a combination of an isolate and a powder.

Preferably, the cosmetically active material includes naturally occurring materials found in eggshell membrane selected from the group consisting of hyaluronic acid, collagen, other proteins, hexosamine, chondroitin sulfate and combinations thereof. The hexosamine can be selected from the group consisting of glucosamine, N-acetyl-D-glucosamine, N-acetyl-D-galactosamine, hexoses and mixtures thereof. Preferably, the cosmetically active material includes cosmetically effective amounts of a hexosamine and/or chondroitin sulfate.

In another aspect, the invention is directed to a composition for use with mammals, which includes naturally occurring material derived from eggshell membrane. The naturally occurring material can be selected from the group consisting of a hexosamine, chondroitin sulfate, hyaluronic acid, collagen, other proteins and combinations thereof.

In one embodiment, the composition is an ingredient in a cosmetic or therapeutic composition. Preferably, the composition is an active ingredient in a cosmetic or therapeutic composition.

In another embodiment, the composition is a topically applied composition for healing wounds or an active ingredient in a topically applied composition for healing wounds.

In yet another embodiment, the composition also includes at least one active ingredient derived from another source. This active ingredient is preferably derived from a marine source. More preferably, it is an isolate or an extract from tissue of an invertebrate marine animal. Most preferably, the active ingredient is an isolate or an extract from tissue selected from the group consisting of mussel, shark cartilage, jellyfish and combinations thereof.

The active ingredient derived from another source can be an ingredient selected from the group consisting of absorbents, anti-acne actives, anti-caking agents, anti-cellulite agents, anti-foaming agents, anti-fungal actives, anti-inflammatory actives, anti-microbial actives, anti-oxidants, antiperspirant/deodorant actives, anti-skin atrophy actives, anti-viral agents, anti-wrinkle actives, artificial tanning agents and accelerators, astringents, barrier repair agents, binders, buffering agents, bulking agents, chelating agents, colorants, dyes, enzymes, essential oils, film formers, flavors, fragrances, humectants, hydrocolloids, light diffusers, nail enamels, opacifying agents, optical brighteners, optical modifiers, particulates, perfumes, pH adjusters, sequestering agents, skin conditioners/moisturizers, skin feel modifiers, skin protectants, skin sensates, skin treating agents, skin exfoliating agents, skin lightening agents, skin soothing and/or healing agents, skin thickeners, sunscreen actives, topical anesthetics, vitamin compounds, and combinations thereof.

In another aspect, the invention is directed to a cosmetic composition containing naturally occurring material derived from eggshell membrane in combination with a cosmetically acceptable carrier. Preferably, the composition is intended for application to the skin or lips. In one embodiment, the cosmetic composition is in a form selected from the group consisting of a powder, liquid, cream, lotion, gel and spray.

Preferably, the natural material derived from eggshell membrane is cosmetically active. The cosmetic composition can also include at least one other component selected from the group consisting of other cosmetically active ingredients, fillers, binders, lubricants, viscosity enhancing agents, fragrances, colorants, visual effect ingredients, other processing agents and combinations thereof.

Preferably, the cosmetically active material is selected from the group consisting of eggshell membrane powder, an eggshell membrane hydrolysate, an eggshell membrane isolate, and combinations thereof.

The cosmetically active material preferably includes naturally occurring material found in eggshell membrane selected from the group consisting of hyaluronic acid, a hexosamine, chondroitin sulfate, collagen, other proteins and combinations thereof.

In one embodiment, the cosmetically active material includes naturally occurring material found in eggshell membrane selected from the group consisting of hyaluronic acid, a hexosamine, chondroitin sulfate and combinations thereof. The cosmetically active material contains at least about 0.2% hyaluronic acid. Preferably, hyaluronic acid is present in an amount of about 0.2 to about 10 wt %, more preferably, about 0.5 to about 5 wt %, and, most preferably, about 1 to about 5 wt %, based on the weight of the cosmetically active material.

The cosmetically active material can include a combination of hyaluronic acid and naturally occurring material selected from the group consisting of a hexosamine, chondroitin sulfate, collagen and other proteins, with the hyaluronic acid being present in a higher percentage than the other naturally occurring material.

In another embodiment, the cosmetically active material includes naturally occurring material found in eggshell membrane selected from the group consisting of collagen, other proteins and combinations thereof. The cosmetically active material typically contains at least about 1% collagen. Preferably, collagen is present in an amount of about 1 to about 40 wt %, more preferably, about 3 to about 25 wt %, and, most preferably, about 5 to about 20 wt %, based on the weight of the cosmetically active material.

The cosmetically active material can include a combination of collagen and naturally occurring material selected from the group consisting of a hexosamine, chondroitin sulfate, hyaluronic acid and other proteins, with the collagen being present in a higher percentage than the other naturally occurring material.

In one embodiment, the carrier is selected to be suitable for application to skin or nails. In this embodiment, the carrier can include one or more compatible liquid or solid filler diluents.

Typically, the carrier is present in an amount from about 75 to about 99.999 wt %, based on the weight of the cosmetic composition. Preferably, the carrier is present in an amount from about 85 to about 99.99 wt %, more preferably, about 90 to about 99 wt % and, most preferably, about 93 to about 98 wt %, based on the weight of the cosmetic composition.

In an embodiment of the invention, the cosmetic composition is an emulsion. The emulsion can be a type selected from the group consisting of oil-in-water, water-in-oil, water-in-oil-in-water, oil-in-water-in-oil, and oil-in-water-in-silicone. Preferably, the material derived from eggshell membrane is an eggshell membrane powder and the composition is an oil-in-water emulsion.

In another embodiment, the cosmetic composition is in a form selected from the group consisting of a cream, wax, paste, lotion, milk, mousse, gel, oil, tonic and spray. The cosmetic composition can be a cosmetic product selected from the group consisting of a hand or body lotion, cold cream, facial moisturizer, anti-acne preparation, topical analgesic, foundation, eyeshadow and lipstick.

The invention is also directed to a cosmetic composition containing a safe and effective amount of a cosmetically active component which comprises naturally occurring cosmetically active material derived from eggshell membrane.

The active material derived from eggshell membrane is preferably selected from the group consisting of eggshell membrane powder, an eggshell membrane hydrolysate, an eggshell membrane isolate and combinations thereof. Preferably, the eggshell membrane hydrolysate or isolate is rich in a naturally occurring material selected from the group consisting of hyaluronic acid, a hexosamine, chondroitin sulfate, collagen, other proteins and combinations thereof.

The active material can also include a combination of an eggshell membrane power and an eggshell membrane hydrolysate or isolate, or a combination of different eggshell membrane hydrolysates or isolates.

In one embodiment, the eggshell membrane hydrolysate or isolate is rich in a naturally occurring material selected from the group consisting of hyaluronic acid, a hexosamine, chondroitin sulfate and combinations thereof. Preferably, the eggshell membrane hydrolysate or isolate is rich in hyaluronic acid.

In another embodiment, the eggshell membrane hydrolysate or isolate is rich in a naturally occurring material selected from the group consisting of collagen, other proteins and combinations thereof. The collagen can be a type selected from the group consisting of Type I collagen, Type V collagen, Type X collagen and combinations thereof.

In another embodiment, the eggshell membrane hydrolysate or isolate is rich in both hyaluronic acid and a naturally occurring material selected from the group consisting of a hexosamine, chondroitin sulfate, collagen and other proteins. Preferably, the ratio of HA to the other naturally occurring materials is in the range of about 10:0.1 to about 0.1:10.

In yet another embodiment, the eggshell membrane hydrolysate or isolate is rich in both collagen and a naturally occurring material selected from the group consisting of hyaluronic acid, a hexosamine, chondroitin sulfate and other proteins. Preferably, the ratio of collagen to the other naturally occurring materials is in the range of about 10:0.1 to about 0.1:10.

The cosmetic composition can be in a form selected from the group consisting of a cosmetic cream, a cosmetic lotion, a shampoo, a hair conditioner, a powder make-up and a colored cosmetic.

Preferably, the active component is present in an amount of from about 0.01 to about 50 wt %, more preferably, from about 0.1 to about 35 wt % and, most preferably, from about 0.5 to about 25 wt %, based on the weight of the cosmetic composition. In a topically applied cosmetic, the cosmetic composition preferably contains a naturally occurring active component derived from eggshell membrane in an amount in excess of 0.5 wt %, more preferably in excess of 0.75 wt % and most preferably in excess of 1 wt %, based on the total weight of the cosmetic composition.

In another aspect, the invention is directed to a therapeutic composition which contains a safe and effective amount of a therapeutically active component, which includes therapeutically active naturally occurring material derived from eggshell membrane. The therapeutic composition can also include a therapeutically acceptable carrier. The composition can be a pharmaceutical or a nutraceutical.

In one embodiment, the carrier is acceptable for oral or parenteral administration to a mammal. In this embodiment, the composition is preferably in a dosage form selected from the group consisting of a tablet, capsule, powder, liquid, suspension and emulsion.

In another embodiment, the carrier is acceptable for topical administration to a mammal. In this embodiment, the composition is preferably in a form selected from the group consisting of a liquid, powder, creme or lotion. The composition can be a topically applied liquid, creme or powder useful for wound healing.

In one embodiment, the therapeutically active component can also include a therapeutically active material derived from other sources.

In another aspect, the invention is directed to a nutraceutical composition containing a naturally occurring material derived from eggshell membrane in combination with a nutraceutically acceptable carrier. Preferably, the material derived from eggshell membrane is therapeutically active and the composition is in a form acceptable for oral or parenteral administration to a mammal. Such a composition can be in a dosage form selected from the group consisting of a tablet, capsule, powder, liquid, suspension and emulsion.

In one embodiment, the therapeutically active material includes naturally occurring material found in eggshell membrane selected from the group consisting of hyaluronic acid, a hexosamine, chondroitin sulfate and combinations thereof.

In an embodiment of the invention, the therapeutically active material contains at least about 0.2% hyaluronic acid. Preferably, hyaluronic acid is present in an amount of about 0.2 to about 10 wt %, more preferably, about 0.5 to about 5 wt %, and, most preferably, about 1 to about 5 wt %, based on the weight of the therapeutically active material.

In another embodiment, the therapeutically active material contains at least about 0.2% hexosamine. Preferably, hexosamine is present in an amount of about 0.2 to about 20 wt %, more preferably, about 0.5 to about 10 wt %, and, most preferably, about 2 to about 5 wt %, based on the weight of the therapeutically active material. Preferably, the hexosamine is selected from the group consisting of glucosamine, N-acetyl-D-glucosamine, glucosamine hydrochloride, glucosamine sulfate and mixtures thereof.

In yet another embodiment, the therapeutically active material contains at least about 0.1 wt % chondroitin sulfate. Preferably, chondroitin sulfate is present in an amount of about 0.1 to about 10 wt %, more preferably, about 0.2 to about 5 wt %, and, most preferably, about 0.3 to about 3 wt %, based on the weight of the therapeutically active material.

In one embodiment, the nutraceutical composition also includes therapeutically active material derived from other sources. The therapeutically active material derived from other sources can be material derived from a marine source. The material is preferably an isolate or an extract from tissue of an invertebrate marine animal. More preferably, the isolate or extract from tissue of an invertebrate marine animal is selected from the group consisting of an isolate or extract from a mussel, an isolate or extract from shark cartilage, an isolate or extract from jellyfish, and combinations thereof.

In one embodiment, the nutraceutical composition preferably includes about 50 wt % to about 95 wt % of eggshell membrane powder and about 5 wt % to about 50 wt % of an isolate or extract from tissue of an invertebrate marine animal, or about 5 wt % to about 50 wt % of an isolate or extract prepared from eggshell membrane, based on the total weight of the composition.

In an embodiment of the invention, the naturally occurring material derived from eggshell membrane preferably includes the following:
hyaluronic acid—about 1 to about 5 wt %;
hexosamine—about 2 to about 5 wt %; and
chondroitin sulfate—about 0.3 to about 3 wt %.

The isolate or extract from tissue of an invertebrate marine animal is preferably a jellyfish isolate or extract.

Preferably, the naturally occurring material derived from eggshell membrane is a therapeutically active material in a form selected from the group consisting of eggshell membrane powder, an eggshell membrane hydrolysate, an eggshell membrane isolate, and combinations thereof.

In another aspect, the invention is directed to a nutraceutical composition containing a safe and effective amount of a therapeutically active component which includes naturally occurring therapeutically active material derived from eggshell membrane. The active material derived from eggshell membrane is preferably selected from the group consisting of eggshell membrane powder, an eggshell membrane hydrolysate, an eggshell membrane isolate and combinations thereof.

In one embodiment, the active material is an eggshell membrane hydrolysate or isolate which is rich in a naturally occurring material selected from the group consisting of hyaluronic acid, a hexosamine, chondroitin sulfate and combinations thereof. Preferably, the eggshell membrane hydrolysate or isolate is rich in hyaluronic acid.

The eggshell membrane hydrolysate or isolate can also be rich in both hyaluronic acid and a naturally occurring material selected from the group consisting of a hexosamine and chondroitin sulfate. Preferred ratios are approximately in the range of from about 1:2:0.3 to about 3:5:2 parts hyaluronic acid to hexosamines to chondroitin sulfate.

In one embodiment, the active material includes a combination of an eggshell membrane power and an eggshell membrane hydrolysate or isolate, or a combination of different eggshell membrane hydrolysates or isolates.

Preferably, the active component is present in an amount of from about 50 wt % to about 100 wt %, more preferably about 70 wt % to about 100 wt % and, most preferably, from about 80 wt % to about 100 wt %, based on the weight of the nutraceutical composition. The nutraceutical composition may be only eggshell membrane powder or combinations of eggshell membrane powder with eggshell membrane hydrolysate or isolate, or combinations of eggshell membrane powder with other natural ingredients, such as marine (jellyfish) extract.

In yet another aspect, the invention is directed to a method for treating, conditioning or improving the appearance of skin, which includes applying to a selected area of skin a composition which includes a naturally occurring ingredient derived from eggshell membrane. The ingredient can be selected from the group consisting of cosmetically active ingredients and therapeutically active ingredients and can include the cosmetic and therapeutic compositions discussed above.

In another aspect, the invention is directed to a method for improving the appearance of wrinkled, lined, dry, flaky, aged or photodamaged skin, which includes applying to an affected area of skin a cosmetic composition (as discussed above), in a amount sufficient to improve the appearance of the affected area of the skin.

The invention is also directed to a method for improving skin thickness, elasticity, flexibility and plumpness. The method includes applying to the skin a cosmetic composition which includes a naturally occurring cosmetically active ingredient derived from eggshell membrane, as discussed above, in an amount sufficient to improve the thickness, elasticity, flexibility and plumpness of skin.

In another aspect, the invention is directed to a method of moisturizing and improving the appearance of skin which involves applying to the skin a cosmetic composition which includes a naturally occurring cosmetically active ingredient derived from eggshell membrane as discussed above, in an amount sufficient to moisturize and improve the appearance of skin.

In yet another aspect, the invention is directed to a method for treating a mammal having a condition that will benefit from the administration of a natural material found in eggshell membrane, the method involving administering to the mammal a composition which includes a naturally occurring therapeutically active material derived from eggshell membrane.

In one embodiment, the condition is a wound and the administering step includes topically applying the composition to the wound, in an amount sufficient to promote wound healing. The wound can be a type of wound selected from the group consisting of full or partial thickness tissue wounds, pressure ulcers, venous ulcers, diabetic ulcers, burns and wounds related to donor sites. The wound can also be a skin wound selected from the group consisting of photodamaged skin, skin keratoses, skin distensae and traumatic skin wounds.

The composition being administered can include the compositions which contain naturally occurring material from eggshell membrane as discussed above. Preferably, the composition contains naturally occurring material which includes the following in weight percent:
hexosamine—about 2 to about 5 wt %;
chondroitin—about 0.3 to about 3 wt %;
hyaluronic acid—about 1 to about 5 wt %; and
collagen—about 5 to about 30 wt %.

In another embodiment, the condition is a condition having an inflammatory component and the administering step includes orally or parentally administering the composition to the mammal, in an amount sufficient to treat the condition. The condition having an inflammatory component can be selected from the group consisting of osteoarthritis; rheumatoid arthritis; rheumatism; bursitis; degenerative spinal disc disease; a degenerative condition causing joint, tendon, ligament or soft tissue pain; and trauma to joints, tendons, ligaments or soft tissue. In this embodiment, the naturally occurring material is preferably selected from the group consisting of a hexosamine, chondroitin and combinations thereof.

Preferably, the hexosamine is present in an amount of about 2 to about 5 wt %, and the chondroitin is present in an amount of about 0.3 to about 2 wt %, based on the total weight of the composition.

In one embodiment, the composition being administered also includes therapeutically active material derived from another natural source. Preferably, the source is a marine source, more preferably an invertebrate marine animal source and, most preferably, an invertebrate marine animal source selected from the group consisting of a mussel, shark, jellyfish and combinations thereof.

As a result of the present invention, extremely useful natural materials containing hexosamine (glucosamine), chondroitin (chondroitin sulfate), hyaluronic acid, collagens and other proteins can be used in compositions and treatments for mammals, i.e., animals and humans.

Although the present invention has been described with reference to hen eggshell membrane, one skilled in the art can easily ascertain the use of eggshell membrane from other fowl including emu, ostrich, etc.

### DETAILED DESCRIPTION

The present invention describes therapeutic, cosmetic, and nutraceutical applications of eggshell membrane, processed eggshell membrane and eggshell membrane isolates.

The composition of eggshell membranes was analyzed from a source of cracked eggs, which included the eggshells with the membrane attached. The eggshell membranes were first separated from the eggshells. Following separation from the shell, the eggshell membrane was tested for amino acid profile and for glucosamine, hyaluronic acid, and chondroitin. Initial results from the Corgenix ELISA assay showed hyaluronic acid concentrations from 5%-10% (50 mg/mL to 100 mg/mL). Samples of crude eggshell membrane were processed to extract and purify hyaluronic acid. Initial results demonstrated that the isolated hyaluronic acid exhibited a relatively low concentration of low molecular weight hyaluronic acid (approximately 50,000-100,000 daltons) using size exclusion chromatography and a refractive index detector. Samples of enzyme hydrolyzed eggshell membrane were tested for hyaluronic acid by the uronic acid assay. Results showed hyaluronic acid concentrations between 0.3% and 2% of the total hydrolysate. This hydrolysate represented enzyme treated eggshell membrane diluted about 1:5 in enzyme solution. Therefore, the total hyaluronic acid content of eggshell membrane was between 1.5%-10% hyaluronic acid. This appears to be the highest level of hyaluronic acid measured in any tissue.

The following Table compares the concentration of hyaluronic acid in eggshell membrane to the concentration of hyaluronic acid in other tissues. (from Laurent, T C, Chemistry & Biology of Extracellular Matrix, Academic Press, Volume 2, Pp 763, 1970).

Hyaluronic acid (HA) concentration
(percent by wet weight)

| Source | HA content |
|---|---|
| Vitreous Humor | 0.002% |
| Adult Skin | 0.03-0.09% |
| Synovial Fluid | 0.14-0.36% |
| Umbilical Cord | 0.3% |
| Rooster Comb | 0.75% |
| Eggshell | 5-10% by ELISA |
| Membrane | assay |

The inventors believe that eggshell membrane is a valuable source for hyaluronic acid. This hyaluronic acid can be used in various applications including cosmetics, eye drops, nutraceuticals, and various other medical applications. Furthermore, an eggshell membrane extract or isolate rich in glucosamine and chondroitin can be useful as a nutraceutical to treat joint pain.

The residue following hyaluronic acid extraction has also been analyzed for hydroxyproline content. Results showed that the wet residue contained 4.5% hydroxyproline. Collagen is composed of about 13% hydroxyproline. Therefore, the total collagen content of the residue is about 35% of the wet weight. This collagen residue may also have important medical and non-medical applications. It is known that eggshell membrane primarily contains Type I collagen. It also contains significant quantities of Type V and Type X collagen. Type X collagen may have application in reducing or preventing tissue mineralization.

Therefore, the present invention contemplates useful products derived from eggshell membrane, which include combinations of naturally occurring components from the membrane, such as combinations of the other useful components discussed above.

Eggshell membrane, processed eggshell membrane and eggshell membrane hydrolysates or isolates contain naturally occurring materials derived from eggshell membrane. These materials can be selected from the group consisting of a hyaluronic acid, hexosamine, chondroitin sulfate, collagen and combinations thereof.

The hexosamine is preferably present in an amount of at least about 0.05, more preferably in the range of about 0.5 to about 10, and most preferably in the range of about 2 to about 5 wt %, based on the weight of the eggshell membrane material.

The chondroitin sulfate is preferably present in an amount of at least about 0.05, more preferably in the range of about 0.1 to about 10, and most preferably in the range of about 0.3 to about 2 wt % based on the weight of the eggshell membrane material.

The eggshell membrane, processed eggshell membrane and eggshell membrane hydrolysates and isolates are preferably free of any animal body components or trace thereof, e.g., animal tissue, blood or body fluid components, which are detrimental or undesirable for the contemplated use of the products or product combinations.

The invention is directed to compositions, and methods of using the compositions, which contain eggshell membrane, processed eggshell membrane, eggshell membrane isolates and/or extracts, and combinations thereof. The eggshell membrane material is obtained by methods that preferably include the step of separating the eggshell membrane from the egg yolk, egg white and eggshell prior to subsequent processing and isolation steps.

Typically, the source of eggshell membrane will be from cracked eggs, where the eggshell membrane is still attached to the eggshell. The eggshell membrane can be separated from the eggshell in any convenient manner. Preferably, the eggshell membrane is separated from the eggshell in the absence of any unwanted substance that would remain in the source material, e.g., the eggshell membrane. Unwanted substances will primarily include calcium carbonate from eggshell residuals. However, small amounts of this calcium source may be beneficial in certain applications, i.e., nutraceuticals.

Methods for separating eggshell membrane from the eggshell can include a purely mechanical manner as, for instance, by rolling and pulling the membranes away from the washed shells after removal of the yoke and albumen of fresh or uncooked eggs. Mechanical methods of separating eggshell membranes from cooked eggs are also contemplated.

A combination of mechanical and chemical means of separating the eggshell membrane from the eggshell can also be used, such as agitating coarsely chopped eggshells containing the adhering membranes in the presence of a dilute acid until the membrane separates from the shell and separating the released membranes from the shells. U.S. Pat. No. 3,194,732 to Neuhauser provides a more detailed discussion of methods for separating eggshell membrane from eggshells, which is incorporated herein by reference.

The method also preferably includes dehydrating the separated eggshell membrane to produce eggshell membrane flakes of various dimensions.

The method also preferably includes powdering the eggshell membrane flakes to produce an eggshell membrane powder with a particle size between 100-500 microns. Powdering is accomplished using standard milling or pulverizing procedures to treat eggshell membrane flakes containing about 10% moisture. Sizing is conducted using a series of screens.

In another method, powdered eggshell membrane is subjected to enzymatic hydrolysis using a yeast enzyme. The resulting slurry contains a soluble fraction containing predominantly hyaluronic acid and other aqueous soluble fractions and an insoluble fraction containing predominantly collagens and other insoluble fractions.

In another method, the powdered eggshell membrane is subjected to enzymatic hydrolysis using other protein hydrolyzing enzymes including pronase, pepsin, ficin, papain, and chymopapain, and combinations thereof. The resultant slurry contains a fraction predominantly composed of hyaluronic acid, soluble collagen and other soluble fractions, while the insoluble fraction contains collagens and other insoluble fractions. The insoluble collagen fractions can subsequently be subjected to additional steps to provide pure collagen fractions.

It is preferred that the eggshell membrane isolates, extracts and hydrolysates are prepared without substantially altering the natural ingredients found in the eggshell membrane. Preferably, the ingredients found naturally in the eggshell membrane which have properties useful for cosmetics, nutraceuticals and pharmaceuticals, e.g., hyaluronic acid, collagens, other proteins, hexosamines and chondroitin sulfate, are not substantially altered as a result of the processes used to prepare the isolates, extracts or hydrolysates.

By the terminology “natural material,” “naturally occurring material” or “naturally occurring active material” derived from eggshell membrane is intended material derived from eggshell membrane which contains a significant amount of at least one ingredient or component of the eggshell membrane that is substantially unaltered from the untreated or unprocessed eggshell membrane, in terms of its function as an ingredient useful for cosmetics, nutraceuticals or pharmaceuticals. By substantially unaltered is meant that the selected or desired ingredient(s) or component(s) substantially retain(s) its/their physical characteristics and is/are not significantly decomposed, digested or cleaved. However, other components or ingredients may be altered in certain isolates or hydrolysates. For example, hydrolysates prepared by enzyme treatment may result in naturally occurring proteins being at least partially digested. Preferably, the majority of the naturally occurring ingredients found in the eggshell membrane are substantially unaltered and, more preferably, substantially all of the naturally occurring ingredients are substantially unaltered. Although the physical characteristics of individual components of the eggshell membrane remain substantially unaltered, the overall composition or amounts of different components can be altered depending on the desired composition for a particular isolate, extract or hydrolysate.

In one aspect, the invention is directed to compositions which include naturally occurring materials derived from eggshell membrane. The occurring materials can include any materials found in the eggshell membrane that have medical or non-medical uses for mammals. Preferred materials include materials selected from the group consisting of hyaluronic acid, collagens, hexosamine, chondroitin sulfate and combinations thereof.

Thus, in one aspect, the invention is directed to compositions for use with mammals, including both humans and animals. The compositions include naturally occurring constituents derived from eggshell membrane that are useful to humans or animals. These constituents can include hyaluronic acid, collagens, hexosamine, e.g., glucosamine, and/or chondroitin sulfate.

By the terminology “use with mammals,” is intended treatment of a mammal having any condition, which would benefit from the administration of eggshell membrane preparation or eggshell membrane isolates or combinations of any naturally occurring constituents from the eggshell membrane, as well as inclusion of the composition in any product intended for use by mammals. Uses of the composition can include use as an ingredient in cosmetics, nutraceuticals or pharmaceuticals. Preferred uses include use as a lubricant or moisturizing agent in cosmetics or eye drops, an orally administered nutraceutical or a locally administered composition for treatment of joints afflicted with osteoarthritis. Other uses include use as a vehicle for other pharmacological substances, in wound healing, treatment of periodontal diseases and as an osteoinductive agent. Additional uses for the isolate(s) and/or composition(s) contemplated by the present invention include all known uses for HA, such as those described more fully in U.S. Pat. No. 5,166,331 to della Valle, et al.; U.S. Pat. No. 5,559,104 to Romeo, et al.; and U.S. Pat. No. 5,646,129 to Callegaro, et al.; which are incorporated herein by reference.

In one aspect, the invention is directed to compositions which contain naturally occurring cosmetically or therapeutically active material derived from eggshell membrane. The compositions can include mechanically processed eggshell membrane, such as flakes or powder, or eggshell membrane isolates or extracts. The eggshell membrane isolates can be in liquid, semi-solid or solid form, e.g., partially dehydrated powdered form containing varying amounts of liquid or moisture.

The compositions can contain an eggshell membrane isolate or extract which is processed to be rich in water soluble fractions of the eggshell membrane, e.g., hyaluronic acid, or rich in water insoluble fractions, certain collagens. The isolate or extract can also contain a specific component selected from the group consisting of a hexosamine, chondroitin sulfate, hyaluronic acid, collagen, other proteins and combinations thereof. It is also contemplated that the eggshell membrane isolates or extracts can be processed to be rich in specific type(s) of collagen and/or proteins, depending upon the intended use.

Depending on the specific use, the eggshell membrane isolates or extracts can be tailored to have specific ratios of different components or combinations of components. The compositions can also include combinations of different isolates and/or physically processed eggshell membrane. For example, the compositions can include specific ratios of different isolates or different eggshell membrane powders, or specific ratios of an isolate and a powder.

Preferred uses of the compositions include use as an active ingredient in cosmetic compositions, such as a lubricant or moisturizing agent in cosmetics or as a topically applied composition for healing wounds. It is also contemplated that the compositions can include the naturally occurring material derived from eggshell membrane in combination with other active ingredients, such as pharmaceutically or cosmetically active ingredients from various sources, including marine sources. Examples of such other active ingredients include isolates and extracts from tissues of invertebrate marine animals, such as mussel powder, shark cartilage powder and jellyfish extracts.

Other active ingredients can include ingredients selected from the group consisting of absorbents, anti-acne actives, anti-caking agents, anti-cellulite agents, anti-foaming agents, anti-fungal actives, anti-inflammatory actives, anti-microbial actives, anti-oxidants, antiperspirant/deodorant actives, anti-skin atrophy actives, anti-viral agents, anti-wrinkle actives, artificial tanning agents and accelerators, astringents, barrier repair agents, binders, buffering agents, bulking agents, chelating agents, colorants, dyes, enzymes, essential oils, film formers, flavors, fragrances, humectants, hydrocolloids, light diffusers, nail enamels, opacifying agents, optical brighteners, optical modifiers, particulates, perfumes, pH adjusters, sequestering agents, skin conditioners/moisturizers, skin feel modifiers, skin protectants, skin sensates, skin treating agents, skin exfoliating agents, skin lightening agents, skin soothing and/or healing agents, skin thickeners, sunscreen actives, topical anesthetics, vitamin compounds, and combinations thereof.

Thus, in one aspect, the invention is specifically directed to a cosmetic composition which includes material derived from eggshell membrane, as discussed above, in combination with a cosmetically acceptable vehicle. Preferably, the cosmetic composition is intended for application to the skin. The cosmetic composition can be a powder, liquid, cream, lotion, gel or other cosmetically acceptable medium. The cosmetic composition can also include other cosmetically active ingredients, fillers, binders, lubricants, flow agents, fragrances, colorants, visual effect ingredients or other processing agents.

The cosmetic composition of the present invention preferably includes a cosmetically-acceptable carrier or vehicle for the eggshell membrane material and other optional components. Suitable carriers are well known in the art and are selected based on the end use application. For example, carriers of the present invention include, but are not limited to, those suitable for application to skin. Preferably, the carriers of the present invention are suitable for application to skin (e.g., sunscreens, creams, milks, lotions, masks, serums, etc.) and nails (e.g., polishes, treatments, etc.). Such carriers are well-known to one of ordinary skill in the art, and can include one or more compatible liquid or solid filler diluents or vehicles which are suitable for application to skin and nails. The exact amount of carrier will depend upon the level of the eggshell membrane material and any other optional ingredients that one of ordinary skill in the art would classify as distinct from the carrier (e.g., other active components). The compositions of the present invention preferably comprise from about 75% to about 99.999%, more preferably from about 85% to about 99.99%, still more preferably from 90% to about 99%, and most preferably, from about 93% to about 98%, by weight of the composition, of a carrier.

The carrier and compositions herein can be formulated in a number of ways, including but not limited to emulsions (in emulsion technology, a composition comprises a “dispersed phase” and a “continuous phase;” the dispersed phase existing as small particles or droplets that are suspended in and surrounded by a continuous phase). For example, suitable emulsions include oil-in-water, water-in-oil, water-in-oil-in-water, oil-in-water-in-oil, and oil-in-water-in-silicone emulsions. Preferred compositions for a particulate eggshell membrane component includes an oil-in-water emulsion.

The cosmetic compositions of the present invention, however, are not limited to emulsions and can be formulated into a wide variety of product types, including creams, waxes, pastes, lotions, milks, mousses, gels, oils, tonics, and sprays. Depending upon the intended use, the compositions can preferably be formulated into lotions, creams, gels, and sprays. These product forms may be used for a number of applications, including, but not limited to, hand and body lotions, cold creams, facial moisturizers, anti-acne preparations, topical analgesics, make-ups/cosmetics including foundations, eyeshadows, lipsticks, and the like. Any additional components required to formulate such products vary with product type and can be routinely chosen by one skilled in the art.

If compositions of the present invention are formulated as an aerosol and applied to the skin as a spray-on product, a propellant is added to the composition. Examples of suitable propellants include chlorofluorinated lower molecular weight hydrocarbons. It is also contemplated that other known propellants can be used.

The cosmetic composition contains eggshell membrane powder, and/or eggshell membrane hydrolysates or isolates, and can include other cosmetically active ingredients, as discussed above. Thus, in one aspect, the invention is directed to a cosmetic composition which contains a safe and effective amount of the cosmetically active ingredients.

As used herein, “safe and effective amount” means an amount of a compound, component, or composition (as applicable) sufficient to significantly induce a positive effect (e.g., confer a noticeable cosmetic benefit), but low enough to avoid serious side effects, (e.g., undue toxicity or allergic reaction), i.e., to provide a reasonable benefit to risk ratio, within the scope of sound medical judgment.

As used herein, “cosmetically active ingredient” means a compound, material, and/or active that confers an aesthetic feature to the substrate to which it is applied, which is preferably skin.

In one embodiment, the invention is directed to a cosmetic cream composition containing eggshell membrane powder, and/or eggshell membrane hydrolysate or isolates.

In another embodiment, the invention is directed to a cosmetic lotion composition containing eggshell membrane powder, and/or eggshell membrane hydrolysate or isolates.

Another embodiment of the invention is directed to a shampoo composition containing eggshell membrane powder, and/or eggshell membrane hydrolysate or isolates.

Another embodiment of the invention is directed to a hair conditioner composition containing eggshell membrane powder, and/or eggshell membrane hydrolysate or isolates.

In another embodiment, the invention is directed to a powder make-up composition containing eggshell membrane powder, and/or eggshell membrane hydrolysate or isolates.

In yet another embodiment, the invention is directed to a colored cosmetic composition containing eggshell membrane powder, and/or eggshell membrane hydrolysate or isolates.

The cosmetic composition can contain eggshell membrane powder, and/or eggshell membrane hydrolysates or isolates in an amount of from about 0.01 wt % to 50 wt %, preferably in an amount of from about 0.1 wt % to 35 wt %, and, more preferably, in an amount of from about 0.5 wt % to 25 wt %, based on the weight of the cosmetic composition. For topical application, it is preferred that the cosmetic composition contains greater than 0.5 wt % of the material derived from eggshell membrane, more preferably greater than 0.75 wt % and most preferably greater than 1 wt %, based on the weight of the cosmetic composition.

By the term “cosmetic composition” is intended for the purposes of the present invention any composition or agent for external application to human or animal skin, nails, or hair for the purpose of beautifying, coloring, conditioning, or protecting the body surface containing a safe and cosmetically effective amount of the active eggshell membrane and/or eggshell membrane hydrolysate, extract or isolate. A cosmetically effective amount of such eggshell membrane and/or eggshell membrane hydrolysate or isolate is that amount required to bring about the desired cosmetic effect, with from 0.01 wt % to 50 wt % being preferred, 0.1 wt % to 35 wt % more preferred, and 0.5 wt % to 25 wt % being most preferred. One of ordinary skill in the art to which the present invention pertains can readily determine what constitutes a “cosmetically effective amount” without undue experimentation. The present cosmetic composition can be in any form including for example: a gel, cream, lotion, makeup, colored cosmetic formulations, shampoo, conditioning agent (e.g., hair conditioner), cleanser, toner, aftershave, fragrance, nail enamel, and nail treatment product.

The term “colored cosmetic formulation” is intended for the purposes of the present invention to mean those cosmetics containing pigment including for example eye shadow, lipsticks and glosses, lip and eye pencils, eyeliners, mascara, foundations, rouges, concealers and blush.

The term “conditioning agent” is intended for the purposes of the present invention to mean any agent or composition which exerts a conditioning effect on the body including the skin, hair and/or nails upon external application and includes agents or compositions containing, for example, humectants; emollients; oils including for example mineral oil; proteins including the present collagen; and shine enhancers including for example dimethicone and cyclomethicone. The present conditioning agents may be included in any of the present pharmacological and/or cosmetic compositions.

In another aspect, the invention is directed to a therapeutic composition which includes a naturally occurring material derived from eggshell membrane in combination with a therapeutically acceptable carrier or vehicle.

The term “therapeutic composition” is intended for the purposes of the present invention to mean any composition or agent administered to a mammal that confers a therapeutic effect to the mammal. The therapeutic composition can be administered topically, orally or parenterally to the mammal.

Topically applied therapeutic compositions can include compositions or agents applied externally to the skin, hair, or nails of a human or animal body for therapeutic purposes containing an effective amount of the present eggshell membrane and/or eggshell membrane hydrolysate, extract or isolate. A “therapeutically effective amount” is that amount required to bring about the desired therapeutic effect, with 0.01 wt % to 50 wt % being preferred, 0.1 wt % to 35 wt % more preferred, and 0.5 wt % to 25 wt % being most preferred. One of ordinary skill in the art to which the present invention pertains can readily determine what constitutes a “therapeutically effective amount” without undue experimentation. Examples of pharmaceutical or therapeutic agents or compositions in accordance with the invention include ointments, creams, lotions, gels, solutions, and shampoos. More specific examples include for example, acne treatment preparations including creams, soaps, cleansers, moisturizers, ointments and lotions; anti-aging preparations including creams, cleansers, moisturizers and lotions; anti-dandruff preparations including shampoos and conditioners; antibiotic preparations; sunburn preparations; anti-itch preparations; and anti-fungal preparations.

For topical application of a therapeutic composition containing eggshell membrane hydrolysates, isolates or extracts, it is preferred that the therapeutic composition contains greater than 0.5 wt % of the hydrolysate, isolate or extract, more preferably greater than 0.75 wt % and most preferably greater than 1 wt %, based on the weight of the therapeutic composition.

Thus, the present invention provides compositions (or active ingredients) for use in topically applied therapeutic and cosmetic compositions, typically creams. The therapeutic compositions can be used to prevent radiodermatitis and sunburns; to treat abrasions, chafing, chapping, itching, diaper rash, eczema, dermatitis, and radiodermatitis; and to heal abrasions, burns (including radiation, chemical, and thermal burns), slow healing wounds, and ulcers. The cosmetic compositions can be used to nourish the skin and treat wrinkles and stretch marks on the skin. The present invention also provides a method for obtaining the active ingredient and for preparing the therapeutic and cosmetic compositions containing the active ingredient.

Other uses of the present eggshell membrane preparations include the addition of the preparation into solutions of surfactants, detergents, soaps, and similar formulations for use with treatment of hair and skin as a cosmetic, a cosmetic ingredient, and/or pharmacological agent.

The composition according to the invention can include one or more eggshell membrane hydrolysates or isolates as the primary or sole cosmetically or therapeutically active ingredient. Examples of a hydrolysate or isolate (or combinations of hydrolysates or isolates) include materials containing water soluble fractions of the eggshell membrane, water insoluble fractions of the membrane or specific ratios of such fractions. More specific examples include materials rich in hyaluronic acid, glucosamine or collagen, as described above.

Thus, in one embodiment, the invention is directed to a cosmetic composition which includes a cosmetic base or carrier containing an amount of a mixture of the different components found in natural eggshell membrane in an amount sufficient to moisturize and improve the surface condition of the epidermis.

The cosmetic or therapeutic compositions according to the invention can include other cosmetic or therapeutic ingredients. These other cosmetic or therapeutic ingredients can include:
Humectants to retain moisture and keep cosmetics like creams from drying out.
Dispersants to keep particles suspended throughout liquids such as make-up and prevent them from settling to the bottom of the container.
Surfactants to help liquids such as hand lotions to spread quickly and easily over the surface of skin.
Abrasives as polishing agents such as the fine grit in the texture of toothpaste.
Emulsifiers to keep liquids such as oil and water from separating into two layers in products such as lotions.
Fixatives to stabilize fragrances so that cosmetics will not lose their pleasing aromas.
Absorbent to attract and hold moisture so that cosmetics such as powders feel dry on the skin.
Preservatives to help to keep cosmetics from deteriorating or spoiling by killing bacteria and fungus.
Colors that may be natural although many are synthetic created from petroleum (called coal tar dyes).
Solvents to dissolve ingredients just as ethyl acetate does in nail polish.

The cosmetic compositions in accordance with the invention can be in various forms including lotions, creams, moisturizers, gels, sun screens, makeup, cleansers, soaps, shampoos, hair conditioners, skin firming compositions, protein concentrates, after shaves, colored cosmetics including for example eye shadows and blushes, nail enamels, and so forth. The therapeutic compositions can be in any known form for therapeutic agents or compositions. The term “therapeutic agent” is the same as “therapeutic composition” and means an agent or composition applied externally to the skin, hair, or nails of the human or an animal body for therapeutic purposes. Examples of preferred therapeutic agents or compositions in accordance with the invention include ointments, creams, lotions, gels, soaps, solutions, and shampoos.

The present eggshell membrane preparations or compositions can also be an ingredient which is added to various formulations of skin care products generally described as lotions for application to human facial or body skin. These lotions generally contain from about 20-80% oil and 10-80% water in an emulsion form. In addition, the lotion may contain humectants, emollients, surfactants, fragrances, preservatives, and so forth. About 5-10% humectant, about 5-20% emollient, and about 0.5-10% surfactant are suggested. Eggshell membrane powder preparations (at about 0.01 to 5.00 wt %) and hydrolyzed eggshell membrane (at about 0.1 to 10.0 wt %) products may be incorporated into moisturizing creams. Creams generally contain from about 20-70% water and about 30-70% oil. In addition, creams may contain a variety of humectants, emollients, surfactants, preservatives, and fragrances. About 5-10% humectant, about 5-20% emollient, and about 0.5-10% surfactant are suggested.

Eggshell membrane preparations (about 0.1 to 2.0 wt %) and eggshell membrane hydrolysates (about 0.01 to 5.00 wt %) can be incorporated into colored cosmetics such as eye shadow or blush. For example, a suitable eye shadow comprises 5-40% pigment, 1-50% oil, and 1-20% waxes. Additionally, the composition may contain one or more of 10-60% water, 0.5-30% surfactant, 1-10% humectants, 0.1-5% preservative, and 0.1-20% silicone.

Eggshell membrane preparations (about 0.2 to 2.00 wt %) and eggshell membrane hydrolysates (about 0.2 to 5.00 wt %) products can also be incorporated into shampoos and hair conditioners. Suitable shampoo formulations include 1-40% surfactant and 10-90% water. Suitable hair condition formulations include 30-95% water, 0.5-30% conditioning ingredients including for example, emollients, proteins, and shine enhancers, and 1-40% surfactant. Hair conditioners and shampoos may also contain thickeners and silicone. About 0.05-5% silicone is suggested in shampoos and hair conditioners.

I the preparation of cosmetic or pharmaceutical products using the present invention, eggshell membrane preparations including powder, hydrolysates, and isolates can be used in effective amounts of about 0.01 to 50 wt % of the preparation, with 0.1 to 30 wt % preferred, and 0.5 to 25 wt % most preferred. The eggshell membrane preparations may be incorporated into suitable cosmetic or pharmaceutical vehicles such as lotions, creams, ointments, gels, shampoos, conditioners, or solutions. Suitable ointments are hydrophilic ointments (USP) or petrolatum and cosmetically effective amounts of eggshell membrane preparations are incorporated into the ointment for topical application to skin or hair. Suitable lotions and creams are as mentioned previously for cosmetic compositions.

Suitable humectants for use in the cosmetic compositions of the present invention include for example glycerin, propylene glycol, butylene glycol, urea, sorbitol, sodium PCA, gelatin, polyethylene glycols, sodium lactate, and hyaluronic acid, dipropylene glycol, polypropylene glycol, hydroxypropyl sorbitol, hexylene glycol, 1,3-butylene glycol, 1,2,6-hexanetriol, ethoxylated glycerin, propoxylated glycerin and mixtures thereof. Most preferably the humectant is glycerin. Amounts of humectant may range anywhere from about 1 to about 50%, preferably from about 10 to about 40%, optimally from about 25 to about 35% by weight.

Suitable emollients include for example glyceryl stearate, cetyl alcohol, stearyl alcohol, isopropyl stearate, stearyl alcohol, stearyl stearate, isopropyl stearate, stearic acid, isobutyl palmitate, isocetyl stearate, oleyl alcohol, sebacates, myristates, palmitates, squalenes, glyceryl monooleate, oleic acids, lanolin, acetylated lanolin alcohols, petrolatum, mineral oils, palmitic acids, and isostearyl neopentanoate. Amounts of the emollient may range from about 1 to about 50%, preferably from about 3 to about 25%, optimally from about 5 to about 20% by weight.

A variety of surfactants can be used in the compositions of the invention including amphoteric, anionic, cationic, or nonionic surfactants. Suitable amphoteric surfactants include imidazolines, betaines, and amino acid salts. Suitable anionic surfactants include for example fatty acid soaps, salts of higher alkyl sulfates, n-acyl sarcosinates, salt or phosphates, sulfosuccinate salts, alkyl benzene sulfonates, salts of N-acyl glutamate, and polyoxyethylene alkyl ether carboxylic acids. Cationic surfactants include for example alkyl trimethyl ammonium salts, alkyl pyridinium salts, alkyl quaternary ammonium salts, and polyamine fatty acid derivatives. Nonionic surfactants include for example lipophilics such as sorbitan fatty acid esters, glycerol fatty acids, propylene glycol fatty acid esters; hydrophilics including for example polyoxyethylene sorbitan fatty acid esters, polyoxyethylenc glycerol fatty acid esters, polyoxyethylene fatty acid esters, polyoxyethylene alkyl ethers, pluronics, polyoxyethylene alkyl phenyl ethers, and polyoxyethylene propylene glycol fatty acid esters. Examples of anionic surfactants which can be used, by themselves or in mixtures, within the framework of the present invention, are the salts, in particular alkali metal (e.g. sodium and potassium), ammonium, alkanolammonium, and alkaline earth (e.g. magnesium and calcium) salts, of the following compounds: alkylsulphates, alkyl-ether-sulphates, alkylamido-ether-sulphates, monoglyceride-sulphates, alkylglycerylsulphonates, alkylsulphonates, alkylphosphates, alkylamidosulphonates, alkylarylsulphonates, alpha-olefin-sulphonates, alkylsulphosuccinates, alkyl-ether-sulpho-succinates, alkylamidosulphosuccinates, alkylsulphosuccinates, alkylsulphoacetates, alkyl-ether-phosphates, acylisethionates, and N-acylamino acids such as N-acylsarcosinates, N-acylglutamates and N-acyllaurates. Among other anionic surfactants generally referred to as soaps are the salts of oleic, ricinoleic, palmitic, myristic, lauric and stearic acids. It is also possible to use weakly anionic surfactants such as salts of acyllactylates.

Preservatives can desirably be incorporated into the compositions of this invention to protect against the growth of potentially harmful microorganisms Particularly preferred preservatives are methyl paraben, propyl paraben, butyl paraben, imidazolidinyl urea, sodium dehydroacetate and benzyl alcohol. The preservatives should be selected having regard for the use of the composition and possible incompatibilities between the preservatives and other ingredients. Preservatives are employed in amounts ranging from about 0.01% to about 2% by weight of the composition. In a preferred embodiment, preservatives (antimicrobials) will be absent from the composition with the exception of pentylene glycol, which has preservative activity.

Minor adjunct ingredients may also be included such as fragrances, antifoam agents, opacifiers and colorants, each in their effective amounts to accomplish their respective functions. Skin active materials may also be formulated with compositions of the present invention. These actives include retinoids such as retinol, retinyl palmitate and retinyl linoleate, alpha-hydroxycarboxylic acids, salicylic acid, potassium glycherrizinate, alpha-bisabolol and combinations thereof. Amounts of these materials may range anywhere from about 0.0001 to about 5% by weight.

Suitable pigments include for example organic and inorganic pigments such as talc, mica, titanium dioxide, titanated mica, iron oxides, ultramarines, chromium oxides, carmine, D&C, and FD&C colors and lakes, ferric and ferrous oxides.

Thus, in a known manner, the composition of the invention may also contain adjuvants which are customary in the cosmetics, such as hydrophilic or lipophilic gelling agents, hydrophilic or lipophilic active agents, preservatives, antioxidants, solvents, perfumes, fillers, screening agents, bactericides, odor absorbers and coloring matter. The amounts of these different adjuvants are those traditionally used in the cosmetic, or dermatological field, and are, for example, from 0.01% to 10% of the total weight of the composition. Those adjuvants, depending on their nature, may be introduced into the fatty phase, into the aqueous phase and/or into lipid spherules.

As oils which can be used in the invention, mineral oils (liquid paraffin), vegetable oils (liquid fraction of shea butter, sunflower oil), animal oils (perhydrosquatene), synthetic oils (Purcellin oil), silicone oils (cyclomethicone) and fluorinated oils (perfluoro polyethers) may be mentioned.

Fatty alcohols, fatty acids (stearic acid) and waxes (paraffin, carnauba, beeswax) may also be used as fatty substances.

As emulsifiers which can be used in the invention, glycerol stearate, polysorbate 60 and PEG/glycol stearate mixtures may be mentioned as examples.

As hydrophilic gelling agents, carboxyvinyl polymers (carbomer), acrylic copolymers such as acrylate/alkylacrylate copolymers, polyacrylamides, polysaccharides such as hydroxypropylcellulose, clays and natural gums may be mentioned, and as lipophilic gelling agents, modified clays such as bentones, metal salts of fatty acids such as aluminum stearates and hydrophobic silica, or alternatively ethylcellulose and polyethylene may be mentioned.

As lipophilic active, agents, retinol (vitamin A) and its derivatives, tocopherol (vitamin E) and its derivatives, essential fatty acids, ceramides and essential oils may be used.

In one aspect, the compositions containing the processed eggshell and/or eggshell hydrolysates or isolates can also include other active ingredients, such as pharmaceutically or cosmetically active ingredients from various sources, including marine animals. Examples of such other active ingredients include isolates and extracts from tissues of invertebrate marine animals, which can be prepared or processed by drying and forming into a powder, as described above with respect to the eggshell membrane. Preferred active ingredients that can be used in combination with the eggshell membrane material include ingredients derived from mussel, shark and jellyfish.

Thus, in another aspect, the invention is directed to a method of treating a mammal having a condition which will benefit from the administration of naturally occurring eggshell membrane substances, including eggshell membrane preparations, eggshell membrane isolates and combinations thereof. Such conditions include, for example, conditions involving dry or wrinkled skin, or stretch marks.

Stretch marks or striae distensae are common blemishes on human skin that usually are even more recalcitrant to treatment than keratoses. Stretch marks are most prevalent on females appearing in the form of white lines or “zebra stripes” and are a rather frequent complication of pregnancy. As the name suggests, the marks are commonly believed to result from the excessive stretching of the skin caused by rapid weight gain, although changes in the levels of various glucocortical hormones have also been implicated.

Normally, the skin is elastic and pliable and can adjust to increases in body girth. However, when the changes are too rapid, the skin's ability to adjust may be overwhelmed. Histologically stretch marks seem to represent a region of skin with absent or disorganized collagen bundles. Although some of the pregnancy-induced marks diminish or even disappear after pregnancy, the more severe marks are usually permanent.

Stretch marks are also formed during the adolescent growth spurt being found on adolescent males as well as females. Significant changes in weight or body size as with body builders can also induce stretch marks. There has been a considerable effort to provide preparations that either prevent the occurrence of the marks and/or promote their healing. Conventional folk wisdom has suggested that applications of various creams or emollients may have the desired effect, but there is considerable clinical controversy surrounding claims that creams and the like can have any significant effect on stretch marks.

In another aspect, the invention is specifically directed to a pharmaceutical or nutraceutical composition which includes material derived from eggshell membrane, as discussed above, in combination with a pharmaceutically or nutraceutically acceptable vehicle or carrier. Preferably, the pharmaceutical or nutraceutical composition is intended for oral or topical administration. For oral administration, the pharmaceutical or nutraceutical composition can be in the form of any unit dosage vehicle, such as a capsule or tablet, or in the form of powder, liquid, or contained in any convenient food item. For topical administration, the pharmaceutical or nutraceutical composition can be in the form of a powder, cream, lotion, gel, liquid or spray. The pharmaceutical or nutraceutical composition can also include other pharmaceutically or nutraceutically active ingredients, fillers, binders, lubricants, flow agents, colorants, or other processing agents.

In one aspect, the compositions containing the processed eggshell and/or eggshell isolates can also include other active ingredients, such as pharmaceutically or cosmetically active ingredients from various sources, including marine animals. Examples of such other active ingredients include isolates and extracts from tissues of invertebrate marine animals, which can be prepared or processed by drying and forming into a powder, as described above with respect to the eggshell membrane. Preferred active ingredients that can be used in combination with the eggshell membrane material include mussel powder, shark cartilage powder and jellyfish extracts. In one embodiment, the active ingredients contained in the composition include active ingredients derived from eggshell membrane in an amount from about 1 to about 99% and other active ingredients in an amount from about 99 to about 1%. Preferably, the eggshell derived material accounts for about 50 to about 99% of the active ingredients, and, more preferably, about 55 to about 95%.

In another aspect, the invention is directed to methods of treating a mammal having a condition which will benefit from the administration of naturally occurring eggshell membrane substances, including eggshell membrane preparations, eggshell membrane isolates and combinations thereof. Such conditions can include, for example, conditions involving connective tissue injuries or degeneration; conditions involving an inflammatory response, such as osteoarthritis, rheumatoid arthritis or other joint disorders; wounds; and dry or wrinkled skin.

The naturally occurring materials will preferably include materials selected from the group consisting of a hexosamine, chondroitin sulfate, hyaluronic acid, collagen, other proteins and combinations thereof.

The invention is also directed to methods for producing a product for use with mammals which includes eggshell membrane, physically processed eggshell membrane, eggshell membrane isolates and combinations thereof in a product for use with mammals. Preferably, the eggshell membrane is first separated from the egg white, egg yolk and eggshell prior to preparation of mechanically processed eggshell membrane and eggshell membrane isolates. Specific naturally occurring substances can also be purified prior to incorporation into the product for use with mammals.

Although the present invention has been described with reference to hen eggshell membrane, one skilled in the art can easily ascertain the use of eggshell membrane from other fowl including emu, ostrich, etc. Furthermore, in some examples the present application has been described with reference to a method for eggshell membrane enzyme hydrolysis and subsequent extraction and purification of naturally occurring eggshell membrane substances. One skilled in the art can easily ascertain various methods for eggshell membrane hydrolysis, and for extraction and purification of naturally occurring components (i.e., hyaluronic acid, glucosamine, chondroitin sulfate, collagens) from eggshell membrane sources. Such equivalents are intended to be encompassed in the scope of the present invention. The present application also describes the analysis of eggshell membrane and eggshell membrane residues following hyaluronic acid extraction. This residue also contains a high content of collagen, as determined by measurement of hydropxyproline. One skilled in the art can appreciate the use of this high collagen containing material, alone or in combination with the hyaluronic acid isolates, for medical and non-medical applications.

### EXAMPLES

The following non-limiting examples have been carried out to illustrate preferred embodiments of the invention.

### Example 1

Hen eggshells and attached eggshell membrane (ESM) were obtained from an egg breaking facility. The eggshell membrane was first separated from eggshells. Eggshell membrane was partially dehydrated, collected and immediately packaged in plastic bags and placed in storage. Samples of eggshell membrane were later retrieved for composition analyses. Results of these analyses are shown in Tables 1-3.

TABLE 1
Typical Analysis of Dry Eggshell Membrane

| Component | % ESM |
|---|---|
| Water | 13.9 |
| Protein | 82.2 |
| Fat | 2.7 |
| Carbohydrate | 0.6 |
| Ash (calcium¹⁷) | 0.6 |

TABLE 2
Typical Amino Acid Composition of Eggshell Membrane Protein

| Amino Acid | % |
|---|---|
| Lysine | 2.88 |
| Tryptophan | 2.51 |
| Leucine | 3.85 |
| Aspartic Acid | 7.01 |
| Proline | 8.23 |
| Isoleucine | 2.01 |
| Threonine | 4.42 |
| Glycine | 3.99 |
| Histidine | 2.79 |
| Arginine | 5.33 |
| Tyrosine | 1.33 |
| Glutamic Acid | 8.23 |
| Cystine | 6.01 |
| Alanine | 2.00 |
| Methionine | 2.85 |
| Valine | 5.13 |
| Phenylalanine | 1.48 |
| Serine | 4.28 |

TABLE 3
Typical Constituents of Eggshell Membrane

| Constituent | % |
|---|---|
| Collagens¹⁸ | 35 |
| Glucosamine | 10 |
| Chondroitin | 9 |
| Hyaluronic acid | 5-10 |

### Example 2

The following example relates to the preparation of ESM flakes and powder. Hen eggshells and attached eggshell membrane were obtained from an egg breaking facility. The eggshell membrane was first separated from eggshells. Eggshell membrane flakes were collected and immediately packaged in plastic bags and placed in storage. Powdering was accomplished using standard milling or pulverizing procedures to treat eggshell membrane flakes containing about 10% moisture. The powder was subsequently sized by screening the pulverized powder through a series of calibrated screens to produce a particle size range from 100-500 microns.

### Example 3

The following example relates to the preparation of eggshell membrane isolates. Samples of eggshell membrane were subjected to enzyme hydrolysis using a yeast enzyme complex. The insoluble residue was allowed to gravity settle and the resultant yellowish, clear solution collected. The hydrolysate was analyzed for uronic acid using the carbazole colorimetric assay. Hydrolysate was shown to contain between 0.1-0.3% HA. Since eggshell membrane samples were diluted approximately 1:10, HA content was between 1-3% HA. The insoluble residue was collected and analyzed for collagen, glucosamine and chondroitin concentrations. The residue was high in hydroxyproline indicating a high concentration of collagen. The clear supernatant was stored at refrigerated temperatures.

### Example 4

The following example relates to the preparation and evaluation of eggshell membrane supplemented cremes for cosmetic application on humans. About 7 parts of the eggshell membrane hydrolysate prepared as in Example 3, was mixed with about 93 parts glycerin and applied to an individuals face. The individual reported that wrinkles were reduced and disappeared, plus the skin became softer and more resilient.

### Example 5

The following example relates to the preparation and evaluation of eggshell membrane supplemented cremes for cosmetic application on humans. Eggshell membrane powder prepared as in Example 2 was mixed with glycerin, water, lavender oil and aloe as shown below:

| Ingredient | Amount |
|---|---|
| Glycerin | 40% |
| Water | 20% |
| Eggshell membrane powder | 30% |
| Aloe Vera gel | 9% |
| Essential oil (Lavender oil) | 1% |

This formulation was applied to a forearm of an individual. The contralateral forearm was treated with a commercial ointment. Both compositions were applied daily for 3 weeks. After 3 weeks, the forearm treated with eggshell membrane supplemented crème was visibly smoother, lighter and appeared more pliable. The contralateral forearm still appeared rough, dark and brittle.

### Example 6

A Formula to treat blemished skin is prepared as follows:

| Ingredient | Amount |
|---|---|
| Water | 65% |
| Almond Oil | 8% |
| Natural Jojoba | 12% |
| Cetyl alcohol | 2.5% |
| Sorbitan stearate | 2.5% |
| Polysorbate 60 | 4% |
| Sorbitol | 2% |
| Eggshell membrane powder | 4% |

### Example 7

A simple formulation for treating dry, brittle skin is prepared as follows:

| Ingredient | Amount |
|---|---|
| Aloe Vera Gel | 50% |
| Eggshell membrane powder | 30% |
| Mineral Oil | 19% |
| Essential Oil | 1% |

### Example 8

A formula for use as a body lotion is prepared as follows:

| Ingredient | Amount |
|---|---|
| Water | 59% |
| Almond Oil | 11% |
| Coco Betaine | 11% |
| Cetyl alcohol | 3% |
| Paraben | 1% |
| Propyltrimonium | 6% |
| Copoylmer 28 | 2% |
| Eggshell membrane hydrolysate | 7% |

### Example 9

A simple formulation for treating dry, brittle skin was prepared as follows:

| Ingredient | Amount |
|---|---|
| Mineral oil | 75% |
| Eggshell membrane powder | 25% |

This formulation was applied to the hand and fingers of an individual. The contralateral hand was treated with mineral oil alone. After 2 weeks, the hand treated with eggshell membrane powder appeared smoother. In addition, there was a noticeable reduction in the number and intensity of dark spots in the hand treated with eggshell membrane powder.

### Example 10

The following formulation is prepared as a foundation for dry skin:

| Ingredient | Amount | |
|---|---|---|
| Natural Jojoba | 5 | ml |
| Triglyceride | 5 | ml |
| Pigments | 1-2 | grams |
| Eggshell membrane powder | 1-2 | grams |
| Shea butter | 1.5 | grams |
| Sorbitan stearate | 1 | gram |
| Polysorbate 60 | 2.5 | ml |
| Cetyl alcohol | 0.5 | grams |
| Water | 35 | ml |

### Example 11

The following example relates to the preparation and evaluation of eggshell membrane powder for the treatment of diabetic lesions. Powder as prepared in Example 2 was applied to open sores in a single application, in an amount sufficient to cover the surface of the sore, and within 4 days lesions started to heal.

### Example 12

The following example relates to the preparation and evaluation of capsules for nutraceutical/therapeutic treatments in humans. Eggshell membrane was separated from eggshell by a mechanical method. The separated eggshell membrane was partially dehydrated and powdered using a pulverization mill. The powder was sized to produce powder from approximately 100 microns to approximately 500 microns. This powder was heat treated and placed in size “O” gelatin capsules, containing about 500 mg of eggshell membrane powder. An individual experiencing chronic joint pain volunteered to take one capsule per day. After approximately 3 days, the individual reported a significant reduction in joint pain. The pain returned when the individual stopped taking the capsules.

### Example 13

The following example relates to the preparation and evaluation of isolate for nutraceutical/therapeutic treatments in humans. Eggshell membrane isolates prepared as in Example 3, were filtered through 0.2 micron filters. An individual experiencing joint pain and gout related events volunteered to take aliquots of the eggshell membrane isolate. Aliquots of approximately 1.0 cc were mixed with orange juice and consumed once a day for 5 days. The individual reported a reduction in joint pain and a remission of gout related events.

### Example 14

The following example relates to the preparation and evaluation of isolate for wound healing treatments in humans. Eggshell membrane isolates prepared as in Example 3, were filtered through 0.2 micron filters and stored at refrigeration temperatures. An individual experiencing minor skin lacerations volunteered to administer the sterile filtered eggshell membrane isolate to the wounds. The isolate was applied using a cotton swab. The individual reported rapid healing of the skin laceration with obvious wound contracture in 24 hours. Similar wound contracture was not observed in untreated wounds in this time period.

### Example 15

The following example relates to the preparation and evaluation of eggshell membrane powder to treat joint pain in horses and dogs. Eggshell membrane powder prepared as in example 2, were placed in capsules containing 500 mg of eggshell membrane powder and administered orally to the animals. Both the horse and the dog were seen to move more easily and be in less pain.

### Example 16

The following example relates to the preparation and evaluation of eggshell membrane isolates, eggshell membrane powder, and combinations for treating skin defects and wounds in horses. Eggshell membrane powder prepared as in example 2 was administered to several different types of wounds on different horses. In some cases the eggshell membrane powder was mixed with glycerin and in others it was applied as a dry powder. Wounds appeared to heal more quickly than normal with no scarring and hair grew over the wound area.

### Example 17

The following example relates to the preparation and evaluation of eggshell membrane powder for treating fibromyalgia and muscle spasm. Eggshell membrane isolate prepared as in example 3 was mixed with 500 mg of eggshell membrane powder as prepared in Example 2 and administered orally to an individual suffering from fibromyalgia. After 3 days, the individual reported spasms stopped and pain disappeared.

### Example 18

The following example relates to the preparation and evaluation of eggshell membrane isolates, eggshell membrane powder, and combinations for functional foods in humans. Eggshell membrane is prepared in a powder or liquid form to add to beverages or solid prepared foods as a performance enhancer, for anti-aging properties, or for the treatment or internal or external wounds.

### Example 19

The following example relates to the preparation and evaluation of eggshell membrane isolates, eggshell membrane powder, and combinations for functional foods for animals. Eggshell membrane is prepared in a powder or liquid form to add to feed of animals, specifically, horses, dogs and cats in an old animal formula to improve the animals joint mobility, improve their eyesight and coats and generally to act as an anti aging formula.

### Example 20

The following example relates to the preparation and evaluation of composites of eggshell membrane powder and marine extracts. Eggshell membrane powder prepared as in Example 2 was mixed with jellyfish extract solution. Mixtures containing 50% eggshell membrane powder and 50% jellyfish extract were prepared and dried. The mixed powder was then sieved to provide a consistent powder size and placed in gelatin capsules. The final composition contained 89% eggshell membrane powder and 11% jellyfish extract. A person suffering from pain associated with arthritis took one capsule each day for 7-days. At day 5, the arthritic pain was diminished. The pain did not return as long as capsules were continued.

### Example 21

A person suffering from fibromyalgia reported diminished pain and increased range of motion 3 days after consuming capsules containing a composite formulation of 50% eggshell membrane powder and 50% jellyfish extract. Biochemical analysis showed the composite contained 8.3 mg/gm of hyaluronic acid and 4.0 mg/gm chondroitin.

### Example 22

A person suffering pain associated with a back injury reported alleviation of pain 3 days after consuming capsules containing a formulation of 90% eggshell membrane powder and 10% jellyfish extract. Biochemical analysis showed the composite contained 6.44 mg/gm of hyaluronic acid and 1.4 mg/gm chondroitin.

All references, including patents, publications, and patent applications, mentioned in this specification are herein incorporated by reference in the same extent as if each independent publication, patent or patent application was specifically and individually indicated to be incorporated by reference.

Thus, while there has been disclosed what is presently believed to be the preferred embodiments of the invention, those skilled in the art will appreciate that other and further changes and modifications can be made without departing from the scope or spirit of the invention, and it is intended that all such other changes and modifications are included in an are within the scope of the invention.

### REFERENCES

¹Wu, T-M, et. al., Matrix Biology, 14:507-513, 1994
²From “Egg Science and Technology” Eds. W. J. Stadelman and O. J. Cotterill, Food Products Press, a subsidiary of The Haworth Press, Bingham, N.Y., 1990
³Britton, W. M. and hale, K. K., Poultry Science, 56:865-871, 1977
⁴Baker, J. R. and Balch, D. A., Biochem. J., 82: 352-361, 1962
⁵Harris, E. D., Blount, J. E. and Leach, R. M., Science, 200: 55-56, 1980
⁶Wong, M, et. al., Dev. Biology, 104:28-36, 1984
⁷Arias, J. L., et. al., Connective Tissue Research, 26: 37-45, 1991; Aria, J. L., et. al., Matrix, 11: 313-320, 1991
⁸Arias, J. L., et. al., Connective Tissue Research, 36: 21-33, 1997
⁹Baker, J. R. and Balch, D. A., Biochem. J., 82: 352-361, 1962
¹⁰Picard, J., Paul-Gardais, A., and Vedel, M, Biochimica et Biophysica Acta, 320: 427-441
¹¹Gautron, J., et. al., Connective Tissue Research, 42:255-267, 2001
¹²Starcher, B. C. and King, G. S., Connective Tissue Research, 8:53-55, 1980
¹³Akagawa, M, et. al., Biochim. Biophys. Acta, 14:151-160, 1999
¹⁴Hincke, M. T., et. al., Matrix Biology, 19:443-453, 2000
¹⁵Cifonelli, J A, “The colorimetric estimation of uronic acid” In: Methodology of Connective Tissue Research, ED: D A Hall, Joynson-Bruvvers Ltd, Oxford, 1976, Chapter 26, Pp 253-256.
¹⁶Armstrong, D C and Johns, M R, “Improved Molecular Weight Analysis of Streptococcal Hyaluronic Acid by Size Exclusion Chromatography” in Biotechnology Techniques.
¹⁷ESM may contain up to 10% calcium
¹⁸Type I and X Collagen (Arias, et. al., Connective Tissue Research, 36: 21-33, 1997; Arias, et. al., Connective Tissue Research, 26: 37-45, 1991) and Type V Collagen (Wong, et. al., Dev. Biol., 104: 28-36, 1984

1-72. (canceled)

73. A nutraceutical composition comprising natural material derived from eggshell membrane in combination with a nutraceutically acceptable carrier.

74. The nutraceutical composition according to claim 73, wherein said material derived from eggshell membrane is therapeutically active.

75. The nutraceutical composition according to claim 74, wherein said composition is in a form acceptable for oral or parenteral administration to a mammal selected from the group consisting of a tablet, capsule, powder, liquid, suspension and emulsion.

76. (canceled)

77. The nutraceutical composition according to claim 74, wherein said therapeutically active material comprises naturally occurring material found in eggshell membrane selected from the group consisting of hyaluronic acid, a hexosamine, chondroitin sulfate and combinations thereof

78. The nutraceutical composition according to claim 77, wherein said therapeutically active material comprises at least about 0.2% hyaluronic acid.

79. The nutraceutical composition according to claim 77, wherein said therapeutically active material comprises at least about 2% hexosamine.

80. The nutraceutical composition according to claim 79, wherein said hexosamine is selected from the group consisting of glucosamine, N-acetyl-D-glucosamine, glucosamine hydrochloride, glucosamine sulfate and mixtures thereof.

81. The nutraceutical composition according to claim 77, wherein said therapeutically active material comprises at least about 0.5% chondroitin sulfate.

82. The nutraceutical composition according to claim 74, wherein said composition further comprises therapeutically active material derived from other sources.

83. The nutraceutical composition according to claim 82, wherein said therapeutically active material derived from other sources is material derived from a marine source.

84. The nutraceutical composition according to claim 83, wherein said material derived from a marine source is an isolate or an extract from tissue of an invertebrate marine animal.

85. The nutraceutical composition according to claim 84, wherein said isolate or extract from tissue of an invertebrate marine animal is selected from the group consisting of an isolate or extract from a mussel, an isolate or extract from shark cartilage, an isolate or extract from jellyfish, an isolate or extract from sea cucumber and combinations thereof.

86. The nutraceutical composition according to claim 85, wherein said nutraceutical composition comprises about 50 wt % to about 95 wt % of natural material derived from eggshell membrane and about 5 wt % to about 50 wt % of an isolate or extract from tissue of an invertebrate marine animal, based on the total weight of the composition.

87. The nutraceutical composition according to claim 86, wherein said natural material derived from eggshell membrane comprises the following:
hyaluronic acid—about 1 to about 5 wt %;
hexosamine—about 2 to about 5 wt %; and
chondroitin sulfate—about 0.3 to about 3 wt %.

88. The nutraceutical composition according to claim 87, wherein said isolate or extract from tissue of an invertebrate marine animal is a jellyfish isolate or extract.

89. The nutraceutical composition according to claim 74, wherein said material derived from eggshell membrane is a therapeutically active material selected from the group consisting of eggshell membrane powder, an eggshell membrane hydrolysate, an eggshell membrane isolate, and combinations thereof.

90. A nutraceutical composition comprising a safe and effective amount of a therapeutically active component which comprises naturally occurring therapeutically active material derived from eggshell membrane.

91. The nutraceutical composition according to claim 90, wherein said active material derived from eggshell membrane is in a form selected from the group consisting of eggshell membrane powder, an eggshell membrane hydrolysate, an eggshell membrane isolate and combinations thereof.

92. The nutraceutical composition according to claim 91, wherein said eggshell membrane hydrolysate or isolate is rich in a naturally occurring material selected from the group consisting of hyaluronic acid, a hexosamine, chondroitin sulfate and combinations thereof.

93. The nutraceutical composition according to claim 92, wherein said eggshell membrane hydrolysate or isolate is rich in hyaluronic acid.

94. The nutraceutical composition according to claim 92, wherein said eggshell membrane hydrolysate or isolate is rich in both hyaluronic acid and a naturally occurring material selected from the group consisting of a hexosamine and chondroitin sulfate.

95. The nutraceutical composition according to claim 91, wherein said active material comprises a combination of an eggshell membrane power and an eggshell membrane hydrolysate or isolate.

96. The nutraceutical composition according to claim 91, wherein said active material comprises a combination of different eggshell membrane hydrolysates or isolates.

97. The nutraceutical composition according to claim 90, wherein said active component is present in an amount of from about 50 wt % to about 100 wt % based on the weight of the nutraceutical composition.

98. (canceled)

99. The nutraceutical composition according to claim 98, wherein said active component is present in an amount of from about 80 wt % to about 100 wt %.

100-124. (canceled)
